(19) United States
(12) Patent Application Publication
(10) Pub. No.: US 2005/0234434 A1
(43) Pub. Date: Oct. 20, 2005

(54) Medical manipulator

(75) Inventor: Takamitsu Sunaoshi, Kanagawa-ken (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 11/082,938

(22) Filed: Mar. 18, 2005

(30) Foreign Application Priority Data
Mar. 30, 2004 (JP) P2004-101130

(51) Int. Cl. A61B 17/00

(52) U.S. Cl. 606/1

(57) ABSTRACT

A medical manipulator including: a rotational assembly configured to transmit driving force; a chassis including a cavity configured to house the rotational assembly; a bearing slot configured to support the rotational assembly; a flush port connecting an outside of the chassis and the cavity; and a channel including openings on a shoulder and a bottom of the bearing slot, the openings being open even if the rotational assembly is fitted into the bearing slot.

### CROSS-REFERENCE TO RELATED APPLICATIONS

This application is based upon and claims the benefit of priority under 35 U.S.C. § 119 from prior Japanese Patent Application P2004-101130 filed on Mar. 30, 2004; the entire contents of which are incorporated by reference herein.

### BACKGROUND OF THE INVENTION

1. Field of the Invention

The present invention relates to a medical manipulator. The present application also relates to a manipulator for use in surgical procedures such as, e.g., laparoscopic cholecystectomy.

2. Description of the Background

Laparoscopic surgery forms three small incisions in the abdominal wall, fits trocars in the incisions, and inserts an endoscope and forceps through the trocars into the abdomen. An operator, usually a surgeon, conducts an operation while watching an image taken by the endoscope and displayed on the screen of a monitor. Laparoscopic surgery reduces physical load on the subject and reduces the number of days for which the convalescent patient is obliged to stay in the hospital before leaving the hospital because laparoscopic surgery does not need to invasively incise the abdominal wall. The field to which such a surgical operation is applicable is expected to expand.

However, considerable technical skills are required of the operator who cannot directly observe the relevant anatomic structures. Moreover, a conventional forceps, comprising only a gripper, lacks operationality for the operation.

To solve this problem, a laparoscopic surgery performed with a forceps controlled with a master-slave method has been considered. In the master-slave method, the operator controls an operating unit with two or more degrees of freedom and the forceps, having several degrees of freedom, acts according to the operation of the operating unit.

A remote controlled type manipulator having an operating unit and a working unit far from each other is an example of a manipulator with the master-slave method. Such a manipulator has many slave-arms arranged near the patient and a master unit arranged far from the patient.

Another example of a master-slave manipulator, and one easier than the remote controlled type to use, is a solid type manipulator having an operating unit and a working unit connected with a common shaft. The solid type manipulator can operate with a simpler system. With the solid type manipulator, the operator operates the manipulator while standing near the patient thereby ensuring a better safety of the patient than with the remote controlled manipulator (see, e.g., JP-A-2000-350735).

The solid type manipulator, however, has a problem with washability. The problem arises because of a complicated structure associated with miniaturization and increase of functions.

Specifically, the solid type manipulator cannot be cleaned inside using an ultrasonic cleaner or a washer-disinfector. Therefore, cleaning the inside of the manipulator is quite problematic. Moreover, this is a serious problem since any blood remaining in the manipulator can lead to a medical malpractice lawsuit.

### BRIEF SUMMARY OF THE INVENTION

The purpose of the invention is to provide a safe medical manipulator having a simple and washable structure.

According to an exemplary embodiment, one aspect of the invention is a medical manipulator including: a rotational assembly configured to transmit a driving force; a chassis including a cavity configured to house the rotational assembly; a bearing slot configured to support the rotational assembly; a flush port connecting an outside of the chassis and the cavity; and a channel including openings on a shoulder and a bottom of the bearing slot, the openings being open even if the rotational assembly is fitted into the bearing slot.

### BRIEF DESCRIPTION OF THE SEVERAL VIEWS OF THE DRAWINGS

The invention and attendant advantages therefore are best understood from the following description of the non-limiting embodiments when read in connection with the accompanying drawings, wherein:

FIG. 1 illustrates a medical manipulator system according to a first embodiment of the invention;

FIG. 2 illustrates the medical manipulator according to the first embodiment of the invention;

FIG. 3 further illustrates the medical manipulator according to the first embodiment of the invention;

FIG. 4 illustrates a block diagram representing a control unit of the medical manipulator system according to the first embodiment of the invention;

FIG. 5 illustrates a casing of the medical manipulator system according to the first embodiment of the invention;

FIG. 6 illustrates a bearing and a bearing slot of the medical manipulator system according to the first embodiment of the invention;

FIG. 7 illustrates a casing of the medical manipulator system according to a first modification of the first embodiment of the invention;

FIG. 8 illustrates a bearing and a bearing slot of the medical manipulator system according to a second modification of the first embodiment of the invention;

FIG. 9 illustrates a casing of the medical manipulator system according to a second modification of the first embodiment of the invention;

FIG. 10 illustrates a casing of the medical manipulator system according to a third modification of the first embodiment of the invention;

FIG. 11 illustrates a casing of the medical manipulator system according to a fourth modification of the first embodiment of the invention;

FIG. 12 illustrates a casing of the medical manipulator system according to a fifth modification of the first embodiment of the invention;

FIG. 13 illustrates a casing of the medical manipulator system according to an eighth modification of the first embodiment of the invention;

FIG. 14 illustrates a casing of the medical manipulator system according to a ninth modification of the first embodiment of the invention;

FIG. 15 illustrates a casing of the medical manipulator system according to a tenth modification of the first embodiment of the invention;

FIG. 16 illustrates a casing of the medical manipulator system according to an eleventh modification of the first embodiment of the invention;

FIG. 17 illustrates a casing of the medical manipulator system according to a twelfth modification of the first embodiment of the invention;

FIG. 18 illustrates the medical manipulator including a knife according to the first embodiment of the invention; and

FIG. 19 illustrates the medical manipulator including a hook according to the first embodiment of the invention.

### DETAILED DESCRIPTION OF THE INVENTION

Referring now to the drawings in which like reference numerals designate identical or corresponding parts throughout the several views.

### First Embodiment

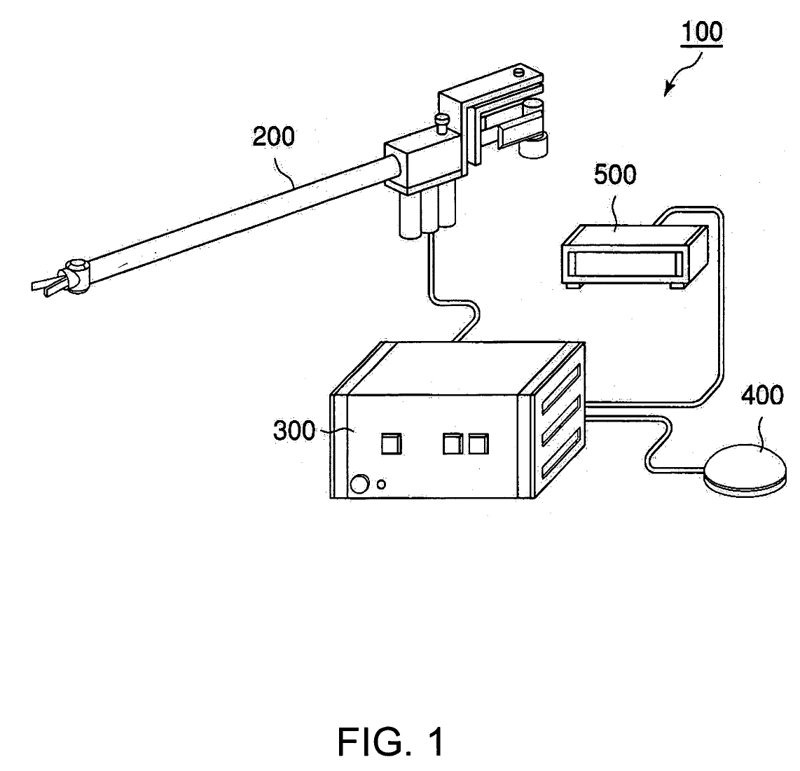

FIG. 1 illustrates an example of a first non-limiting embodiment of a medical manipulator system 100 according to the invention.

The medical manipulator system 100 comprises a medical manipulator 200, a control unit 300, a command interface 400 and a monitor 500.

The command interface 400 receives commands from an operator, which commands may comprise starting work, ending work, or changing an operation mode. For example, in a preferable embodiment of the command interface 400, a foot switch is used by the operator to send the commands without stopping the operation. The command interface 400 could also be a voice recognition device, a hand switch put on the medical manipulator, or any other device allowing the operator to send commands without stopping the operation.

The monitor 500 displays states of the medical manipulator system 100. The monitor 500 may be put near a monitor for displaying a vision from a laparoscope.

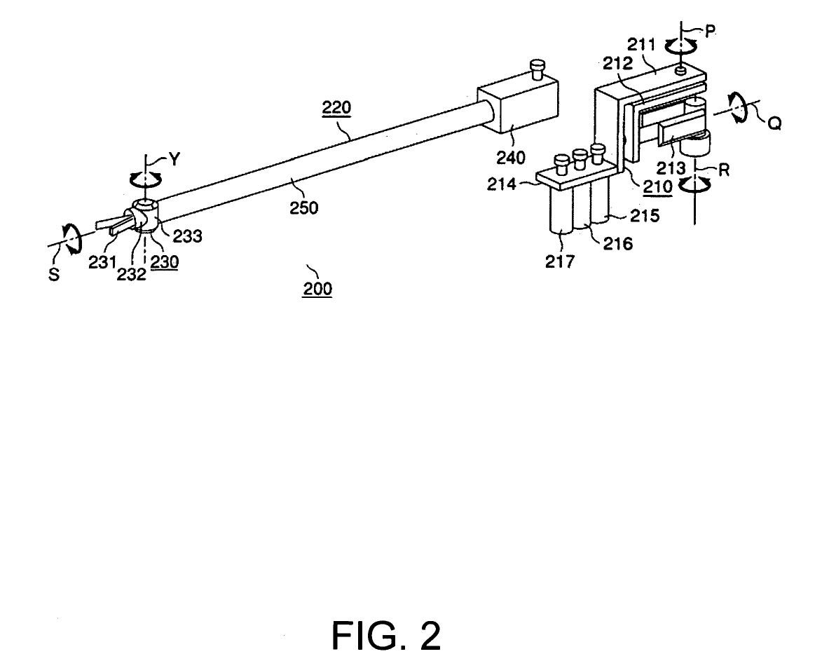

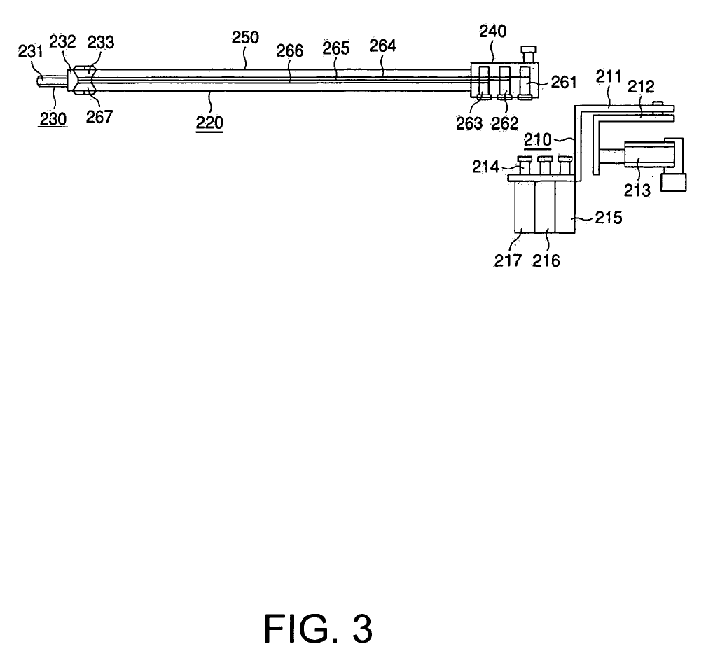

FIG. 2 and FIG. 3 illustrate the medical manipulator 200. The medical manipulator 200 comprises an operating unit 210 and a working unit 220.

The operating unit 210 and the working unit 220 can be combined or separated from each other. The operating unit 210 comprises a first frame 211, a second frame 212, an end effecter control unit 213, a driving unit 214, and motors 215-217.

The second frame 212 is connected to an end of the first frame 211 rotatably about an axis P set on the end of the first frame 211.

The end effecter control unit 213 is connected to the first frame 211 rotatably about an axis Q set on the second frame 212. The axis Q intersects with the axis P. The end effecter control unit 213 has two finger slots 213A and 213B. The finger slot 213A and the finger slot 213B are connected to each other rotatably about an axis R. The axis R is parallel with the axis P.

The driving unit 214, connected to the other end of the first frame 211, has three motors 215-217. The rotation angles of motors 215-217 are controlled by the control unit 300 based on the rotation angle of the second frame 212 about the axis P, the rotation angle of the end effecter control unit 213 about the axis Q and the angle between finger slot 213A and 213B about the axis R.

The second frame 212 has an angle sensor for reading its rotation angle about the axis P.

The end effecter control unit 213 has an angle sensor for reading its rotation angle about the axis Q. The end effecter control unit 213 also has an angle sensor for reading the angle between finger slot 213A and 213B about the axis R.

The working unit 220 comprises an end effecter 230, a casing 240, and a connecting unit 250.

The end effecter 230 is inserted into a patient. Various types of equipment can be included in the end effecter. The end effecter 230 shown in FIG. 2 includes a gripper 231 as an example of the equipment. Furthermore the end effecter 230 includes a roll-free supporting unit 232, a yaw-free supporting unit 233, and a gear set 267.

The gripper 231 has two fingers. These fingers can rotate about the axis Y on one end of each finger. When the gripper 231 performs a grip motion, the other ends of the fingers move closer to one another by rotating about the axis Y. When the gripper 231 performs a release motion, the other ends of the fingers move away from each other by rotating about the axis Y.

The roll-free supporting unit 232 supports the gripper 231 rotatably about the axis S.

The yaw-free supporting unit 233 supports the roll-free supporting unit 232 rotatably about the axis Y.

The gear set 267 transmits the driving force of wires 264-266 to the gripper 231, the roll-free supporting unit 232, and the yaw-free supporting unit 233.

The connecting unit 250, formed like a hollow tube, connects the end effecter 230 and the casing 240.

The casing 240 comprises rotational assemblies 261-263. When the working unit 220 is attached to the operating unit 210, rotational assemblies 261-263 are connected to motors 215-217, respectively. Rotational assemblies transmit the driving forces of motors 215-217 to wires 264-266, respectively.

The end effecter may have various types of equipment including the gripper 231 described above. For example, the end effecter may include two blades of scissors in place of fingers.

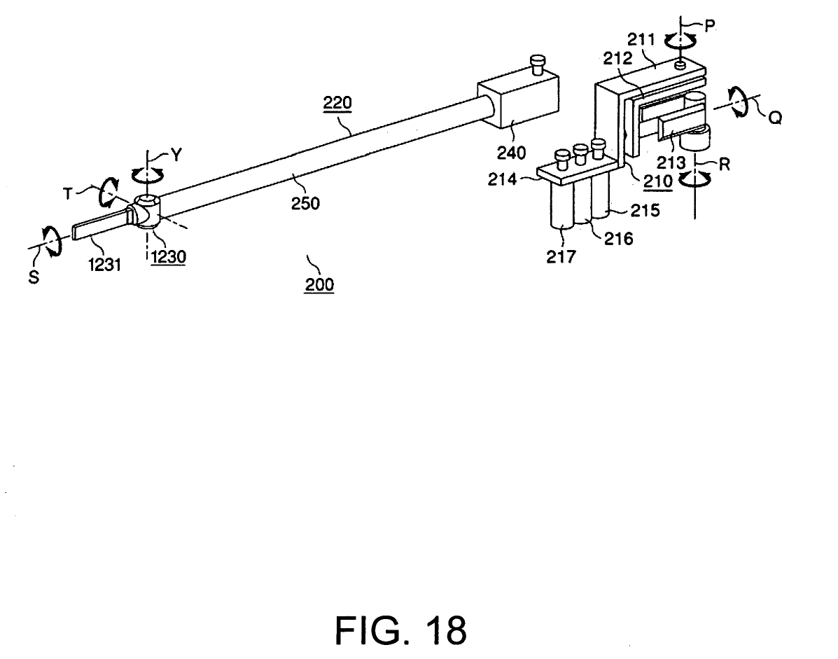

The end effecter 1230 may include a knife 1231 in place of the gripper 231 as shown in FIG. 18. The knife 1231 can be supported rotatably about not only the axis Y and the axis S, but also the axis T. The axis T intersects with the axis Y and the axis S. The knife 1231 may be an electric cautery knife, or a diathermy knife. The knife 1231 may be supported rotatably about only two axes like axis Y and axis T.

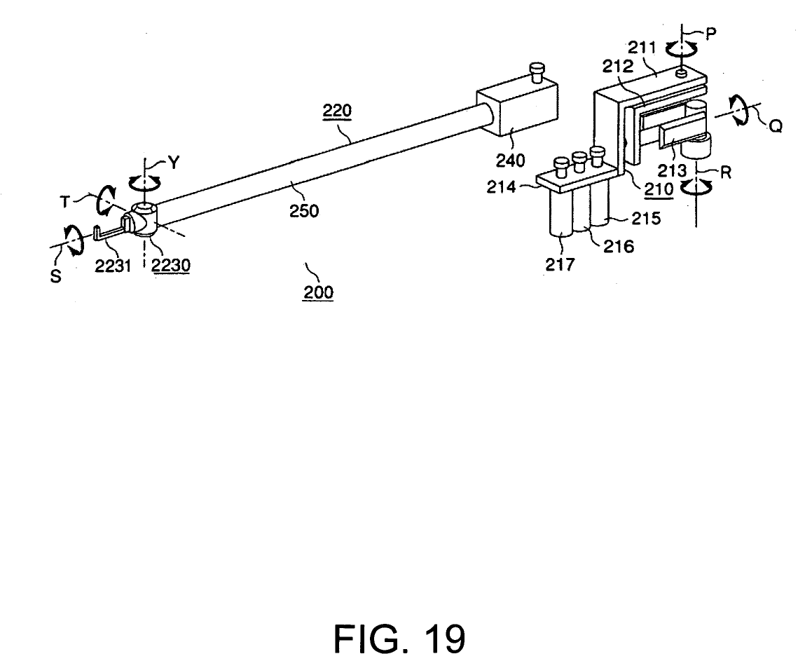

The end effecter 2230 may include a hook 2231 in place of the gripper 231 as shown in FIG. 19. The hook 2231 can be supported rotatably about not only the axis Y and the axis S, but also the axis T. The hook 2231 may be supported rotatably about only two axes like axis Y and axis T.

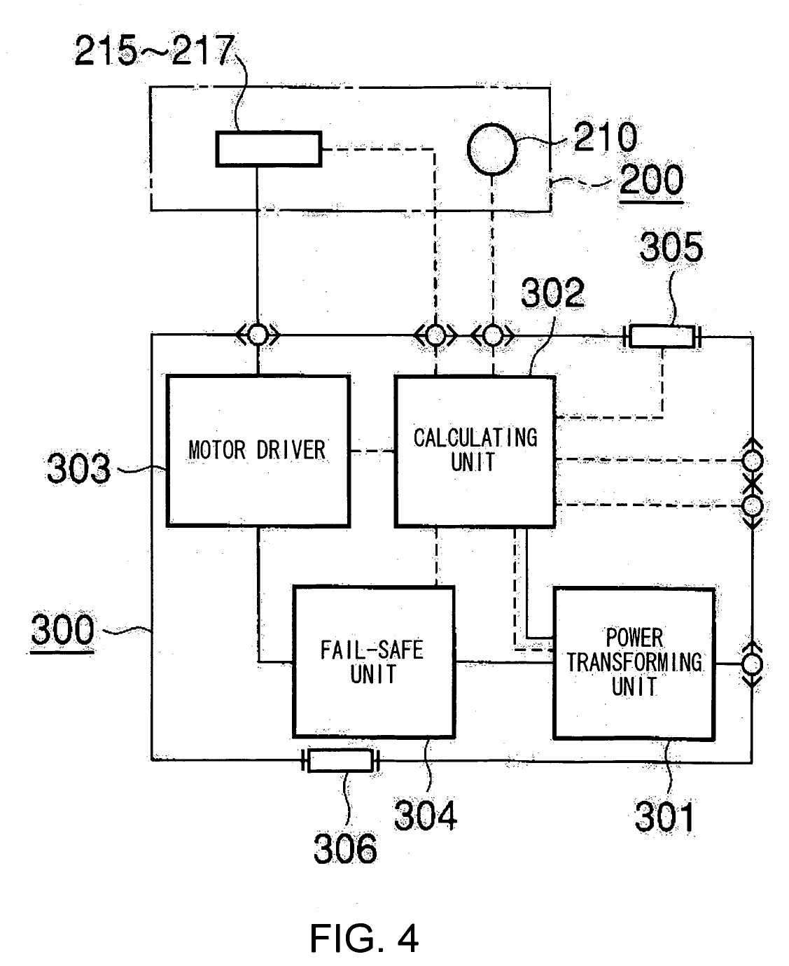

FIG. 4 illustrates a block diagram of the control unit 300. The control unit 300 comprises a power transforming unit 301, a calculating unit 302, a motor driver 303, a fail-safe unit 304, and switches 305 and 306.

The power transforming unit 301 transforms electric power supplied from an outer electric source and supplies the transformed electric power to the calculating unit 302 and the motor driver 303.

The calculating unit 302 comprises, for example, CPUs, memory devices, logic devices, and interfaces. The calculating unit 302 produces control signals based on a target value and provides control signals to the motor driver 303. The calculating unit 302 calculates the target value based on a deviation between the rotating angle of the second frame 212 and the end effecter control unit 213 of the operating unit 210.

The motor driver 303 supplies the electric power to motors 215-217 based on the operation of the calculating unit 302.

The fail-safe unit 304 interrupts the electric power to the motor driver 303 when unusual situations such as, e.g., a stepping out of the operation of the calculating unit 302, an overdrive of the motor driver 303, or a scram instruction, occur.

Switches 305 and 306 are used to change the operation mode and power source.

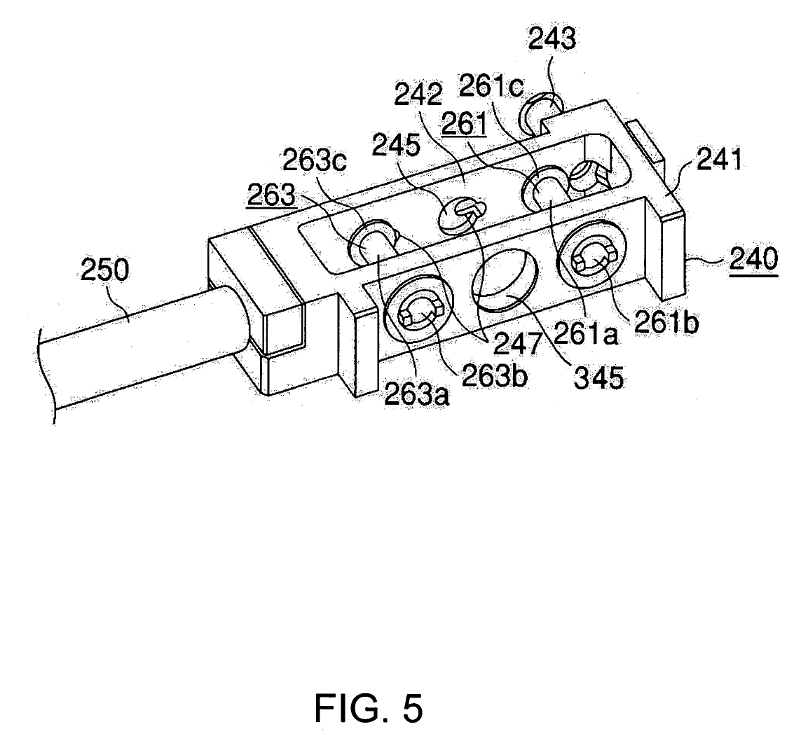

FIG. 5 illustrates a casing 240. One of the walls of the casing 240 is not drawn in FIG. 5. The casing 240 comprises a chassis 241, a cavity 242, and a flush port 243.

The chassis 241 has the cavity 242 in it. The cavity 242 is connected to the hole of the connecting unit 250.

The cavity 242 houses rotational assemblies 261-263. For clarity, only rotational assemblies 261 and 263 are shown in FIG. 5. A wall of the chassis 241 has three pits, facing to the cavity 242, as bearing slots 244-246. Only bearing slot 245 is visible in FIG. 5. Another wall opposite the wall has three holes including bearing slots 344-346. Only bearing slot 345 is visible in FIG. 5.

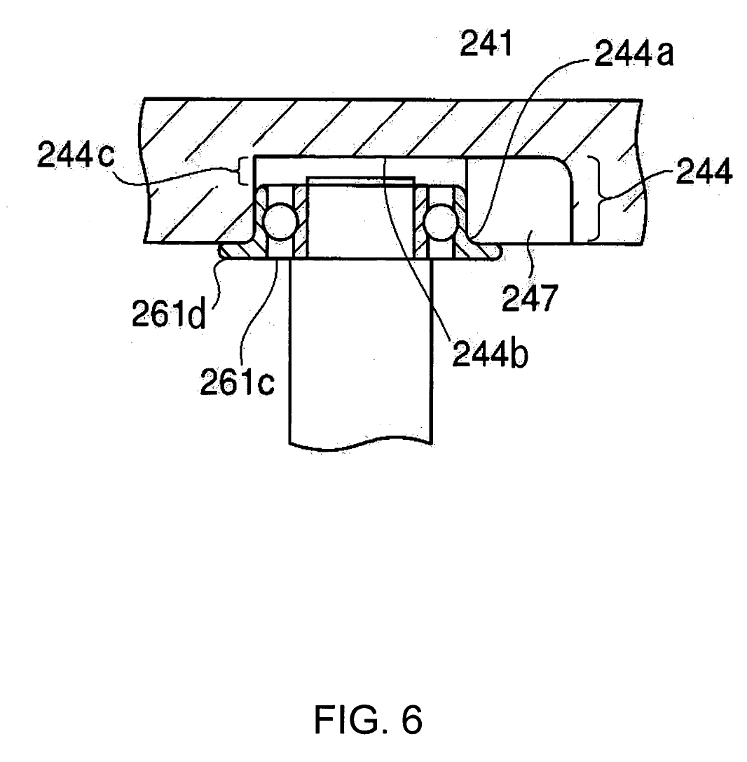

A bearing 261c supporting the rotational assembly 261 is a ball bearing fitted into the bearing slot 244. The bearing 261c has a collar 261d as shown in FIG. 6. The collar 261d of the bearing 261c contacts a shoulder 244a of the bearing slot 244, so there is a gap 244c between the bottom 244b of the bearing slot 244 and the bearing 261c.

The bearing slot 244 has a flushing channel 247. The flushing channel 247 is a channel formed through the shoulder 244a and the bottom 244b of the bearing slot 244, and parallel to a line through center of the bearing slot 244 and the flush port 243.

The flushing channel 247 has openings at least on the shoulder 244a and the bottom 244b.

An opening of the flushing channel 247 on the shoulder 244a is formed at the nearest position to the flush port 243. If the bearing slot 244 is very close to the flush port 243, an opening of the flushing channel 247, which may be on an inside of the flush port 243, connects the flushing channel 247 directly.

The other opening of the flushing channel 247 is formed at the bottom 244b.

The opening of the flushing channel 247 on the shoulder 244a has a depth along a direction perpendicular to the axis of the bearing slot 244 enough to be open to the cavity 242 even if the bearing 261c is fitted into the bearing slot 244. In other words, the collar 261d cannot cover up the opening of the flushing channel 247. The other opening of the flushing channel 247 on the bottom 244b is also open to the gap 244c even if the bearing 261c is fitted into the bearing slot 244. Similarly, a bearing 262c including a collar 262d supporting the rotational assembly 262 (second rotational assembly) is fitted into the bearing slot 245 (second bearing slot). The collar 262d contacts a shoulder 245a of the bearing slot 245, so there is a gap 245c between the bottom 245b of the bearing slot 245 and the bearing 262c. The bearing slot 245 has a flushing channel 247.

A bearing 263c including a collar 263d supporting the rotational assembly 263 is fitted into the bearing slot 246. The collar 263d contacts a shoulder 246a of the bearing slot 246, so there is a gap 246c between the bottom 246b of the bearing slot 246 and the bearing 263c. The bearing slot 245 has a flushing channel 247.

The flush port 243 connects the outside of the chassis 241 and the cavity 242. A washing liquid for flushing the cavity 242 runs into the cavity 242 from the flush port 243. The flush port 243 can be covered with a lid. During a laparoscopic surgery with pneumoperitoneum, air in the cavity 242 can enter and escape from the connecting unit 250.

The rotational assembly 261 comprises a shaft 261a, a clutch 261b, and the bearing 261c.

The clutch 261b is formed on an end of the shaft 261a. The clutch 261b engages with the motor 215.

The bearing 261c is fitted on the other end of the shaft 261a and, as described above, is fitted into the bearing slot 244 of the cavity 242.

Similarly, the rotational assembly 262 comprises a shaft 262a, a clutch 262b, and the bearing 262c. The clutch 262b engages with the motor 216 and is formed on an end of the shaft 262a. The bearing 262c is fitted on the other end of the shaft 262a into the bearing slot 245 of the cavity 242.

The rotational assembly 263 comprises a shaft 263a, a clutch 263b, and the bearing 263c. The clutch 263b engages with the motor 217 and is formed on an end of the shaft 263a. The bearing 263c is fitted on the other end of the shaft 263a into the bearing slot 246 of the cavity 242.

The end effecter 230 of the medical manipulator 200 can be contaminated with blood when the end effecter 230 is inserted into a patient.

Should the end effecter 230 be contaminated with blood, blood will flow into the cavity 242 along wires 264-266 in the connecting unit 250 and reach rotational assemblies 261-263 and bearings 261c-263c. Bearings 261c-263c include a clearance between their inner race and outer race. Therefore, gaps 244c-246c will also be contaminated with blood.

The following describes how blood having reached the gaps 244c-246c can be flushed.

The working unit 220, when detached from the operating unit 210, has no electrical component and can therefore be immersed into a washing liquid in a washing device such as an ultrasonic cleaner or a washer-disinfector.

The bearing slot 244 into which the bearing 261c is fitted has two openings. One of the openings is the clearance between the inner race and outer race of the bearing 261c. The other is the opening of the flushing channel 247 on the shoulder 244a of the bearing slot 244.

These two openings act as inlet and outlet. The washing liquid can flush blood out from the flushing channel 247 thereby washing well not only outside but also inside the working unit 220.

Since the opening of the flushing channel 247 on the shoulder 244a of the bearing slot 244 is formed at the nearest position to the flush port 243, washing liquid can run into the opening easily.

Since the flushing channel 247 is formed parallel to a line through center of the bearing slot 244 and the flush port 243, dynamic pressure of the washing liquid from the flush port 243 can be transferred to the other opening of the flushing channel 247 well.

The working unit 220 can also be washed inside by inpouring washing liquid from the flush port 243 and drawing it off from gaps of structures of the end effecter 230.

The efficacy of this method was ascertained experimentally. The medical manipulator 200 described above was first washed with the washer-disinfector after inpouring blood into the medical manipulator 200. Amido black solution, which reacts with protein, was subsequently applied on bearing slots 244-246. As a result, no stain appeared around the bearing slots 244-246. In other words, protein was flushed away well and so was the blood containing it.

According to this embodiment, the flushing channel 247, including two openings, formed on the bearing slot 244, enables to flush blood out from the gap 247 well.

### FIRST MODIFIED EXAMPLE

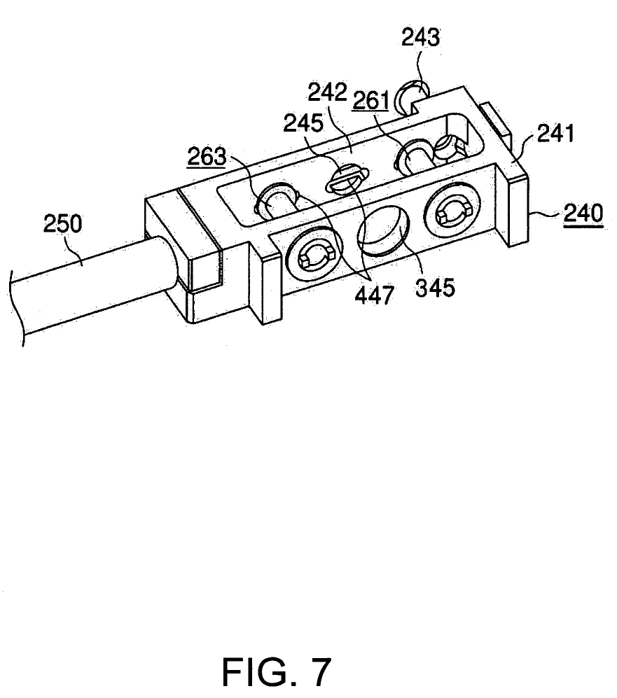

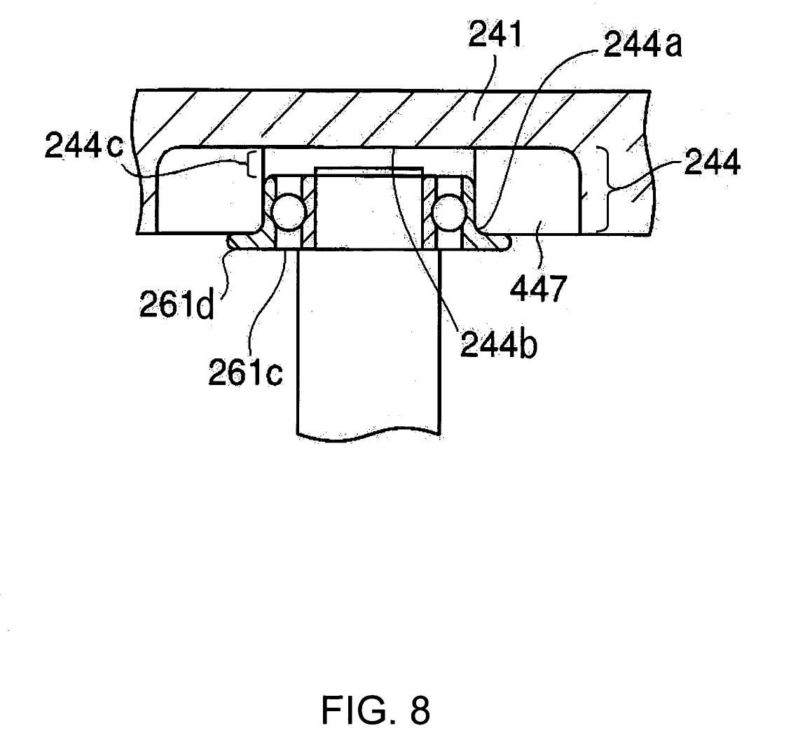

FIG. 7 and FIG. 8 illustrate a first modification of a first non-limiting embodiment of the casing 240.

The flushing channel 447 is a channel formed through the shoulder 244a and the bottom 244b of the bearing slot 244. In this modified example, the flushing channel 447 is through the center of the bearing slot 244 and reaches the other side of the shoulder 244a. The flushing channel 447 thus has at least two openings on the shoulder 244a and one opening on the bottom 244b.

Both openings of the flushing channel 447 are open to the cavity 242 even if the bearing 261c is fitted into the bearing slot 244. In other words, the collar 261d cannot cover up both openings of the flushing channel 447.

According to this modification of the non-limiting embodiment, the flushing channel 447, including two openings on the shoulder 244a of the bearing slot 244, enables flushing blood out from the gap 447 well.

### SECOND MODIFIED EXAMPLE

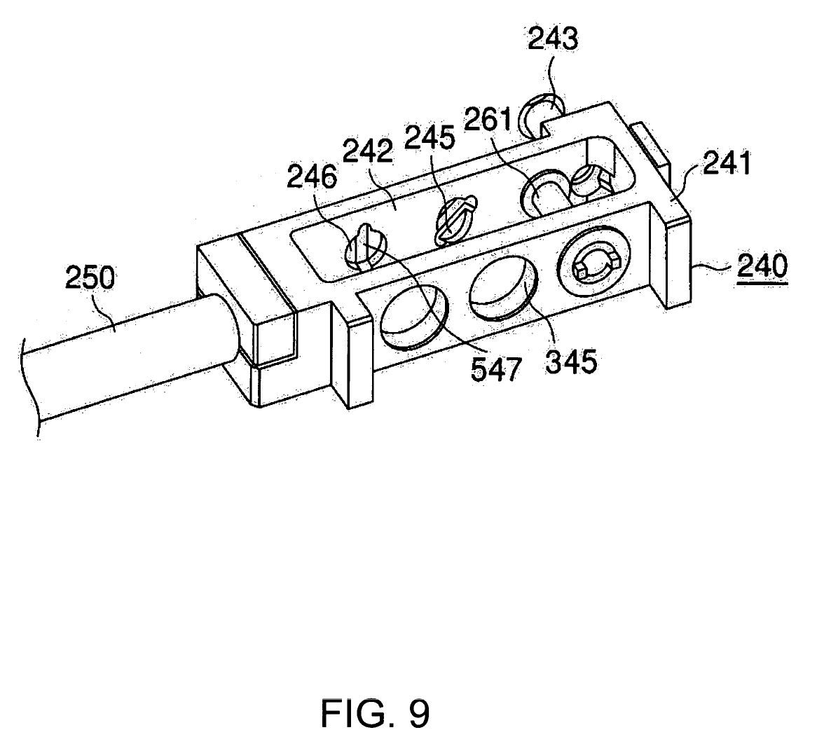

FIG. 9 illustrates a second modification of a first non-limiting embodiment of the casing 240.

In this example, the flushing channel 547 is formed through the center of the bearing slot 246, and perpendicular to a line passing through the center of the bearing slot 246 and the flush port 243.

Openings of the flushing channel 547 on the shoulder 245a of the bearing slot 245 are on a line that crosses the line passing through the center of the bearing slot 246 and the flush port 243 at an angle of 45 degrees at the center of the bearing slot 245.

As described above, the position of the opening of the flushing channel 547 is not limited to the nearest position on the shoulder 246a of the bearing slot 246. The opening of the flushing channel 547 should be arranged at an efficient position for washing with the flow of the washing liquid in the cavity 242. THIRD MODIFIED EXAMPLE

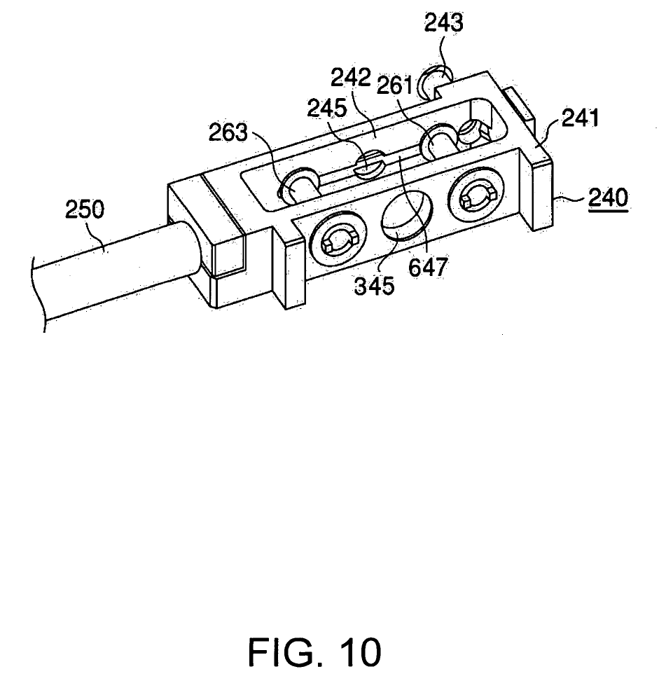

FIG. 10 illustrates a third modification of a first non-limiting embodiment of the casing 240.

In this example, the flushing channel 647 is formed straight through not only the bearing slot 244 but also the bearing slot 245 (second bearing slot) and the bearing slot 246. The opening of the flushing channel 647 connects to the flush port 243.

Because the flushing channel 647 connects to the flushing port 243, dynamic pressure of the washing liquid from the flush port 243 can be transferred to the other opening of the flushing channel 647. The bearing slots 244-246 can thus be washed well.

### FOURTH MODIFIED EXAMPLE

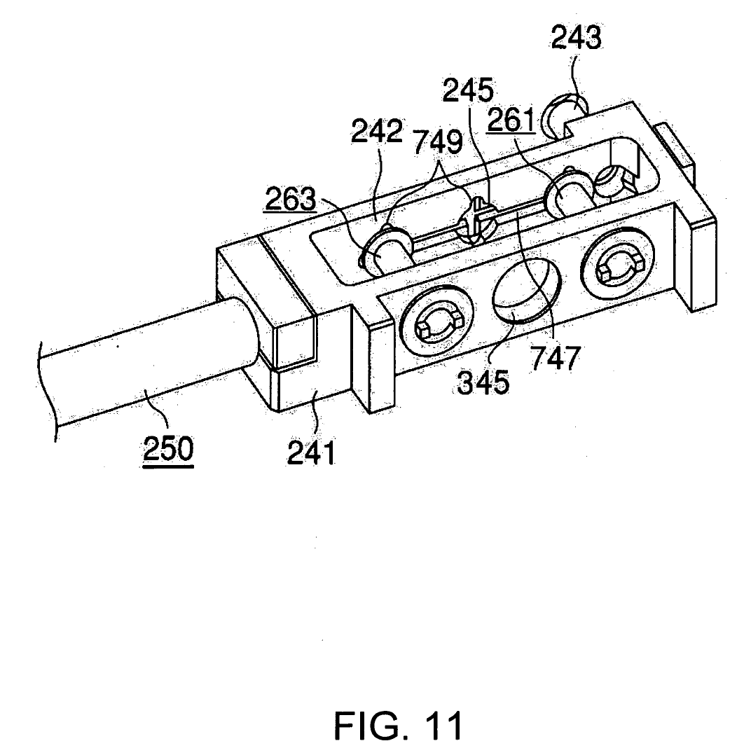

FIG. 11 illustrates a fourth modification of a first non-limiting embodiment of the casing 240.

In this example, the bearing slot 244 has a sub flushing channel 749 cross perpendicular to the flushing channel 747.

The sub flushing channel 749 is through the center of the bearing slot 244 and has at least two openings on the shoulder 244a of the bearing slot 244.

When the working unit 220 is immersed into washing liquid, with the flush port 243 upward, the flushing channel 747 becomes horizontal, and the sub flushing channel 749 becomes vertical. Therefore, air in the bearing slot 244 and the horizontal flushing channel 747 can be flushed out from the opening of the vertical sub flushing channel 749 by the flow of washing liquid so that the bearing slot 244 can be washed well.

Even if the flushing channel 747 and the sub flushing channel 749 are not perpendicular to each other, the flushing channel 747 can be washed well because a number of outlets of the air in the flushing channel 747 increases.

Further, the increased number of air outlets in the flushing channel 747 facilitates drying.

### FIFTH MODIFIED EXAMPLE

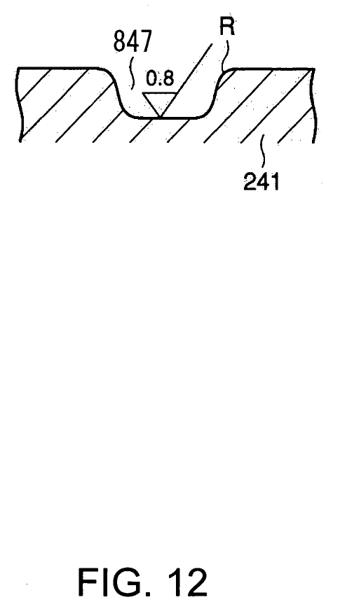

FIG. 12 illustrates a fifth modification of a first non-limiting embodiment of the casing 240. In this example, the surface roughness of at least one surface of the flushing channel 847 is 0.8 micrometers or less. At least an edge of the flushing channel 847 is rounded off. At least a surface of the flushing channel 847 is coated with fluoroplastics.

With the above mentioned configuration, blood cannot be fixed in the flushing channel 847 and the washing liquid can flow in the flushing channel 847 well.

### SIXTH MODIFIED EXAMPLE

A sixth modification of a first non-limiting embodiment of the casing 240 is described below.

In this example, the chassis 241 is made of PEEK (poly ether ether ketone), shafts 261a-263a are made of aluminum, and bearings 261c-263c are made of stainless steel. PEEK has good heat resistance which makes it suitable for a medical appliance that is sometimes sterilized with heat.

An outer diameter of bearings 261c-263c and an inner diameter of bearing slots 244-246 are approximately lomm, and an outer diameter of shafts 261c-263c and inner diameter of bearings 261c-263c are approximately 3 mm.

A washing liquid is typically warmed to about 55 degrees centigrade. The temperature of the chassis 241 is thus warmed from 25 degrees centigrade (ordinary temperature) to 55 degrees centigrade (washing liquid temperature), a temperature difference of about 30 degrees centigrade.

Linear thermal expansion coefficients of PEEK, Aluminum, and stainless steel are 5.0×10−5, 2.3×10−5, and 1.25×10−5, respectively.

Generally speaking, linear thermal expansion coefficient α, temperature change ΔT, length before temperature changes L0, and elongation ΔL have a relation represented by equation (1).
ΔL=α×L₀×ΔT (1)

If the temperature of the chassis 241 increases 30 degrees centigrade, the inner diameter of the bearing slot 244 of the chassis 241 made of PEEK expands 5.0×10⁻⁵×10×30=15.0×10⁻³ mm and the outer diameter of the bearing 261c made of stainless steel expands 1.25×10⁻⁵×10×30=3.75×10⁻³ mm.

An 11 μm gap thus appears between the bearing slot 244 and the bearing 261c.

Originally, there is a very narrow gap between the shaft 261a and the bearing 261c, and between the bearing slot 244 and the bearing 261c, to ensure proper fitting. However, the gap is wide enough for blood to leak into.

Consequently, the shaft 261a is made of a material that has a smaller linear thermal expansion coefficient than that of a material of the bearing 261c. Similarly, the bearing 261c is made of a material that has a smaller linear thermal expansion coefficient than that of a material of the chassis 241.

With the above mentioned configuration, the flow of washing liquid can flush blood in the gap well when washing.

### SEVENTH MODIFIED EXAMPLE

A seventh modification of a first non-limiting embodiment of the casing 240 is described as below.

In this example, the bearing 261c is not a ball bearing but a solid bearing made of PEEK. An outer diameter of shafts 261c-263c and an inner diameter of bearings 261c-263c are approximately 3 mm.

The inner diameter of the bearing 261c made of PEEK therefore expands 5.0×10⁻⁵×3×30=4.5×10⁻³ mm and the outer diameter of the shaft 261a made of aluminum expands 2.3×10⁻⁵×3×30=2.1×10⁻³ mm.

As a result, a 2 μm gap appears between the bearing 261c and the shaft 261a.

With the casing 240 described above, it is desirable for a lubricant to be added to the washing liquid. The lubricant acts not only as a lubricant, but also as a sealer. The lubricant in the gap guards the gap against blood leaking.

### EIGHTH MODIFIED EXAMPLE

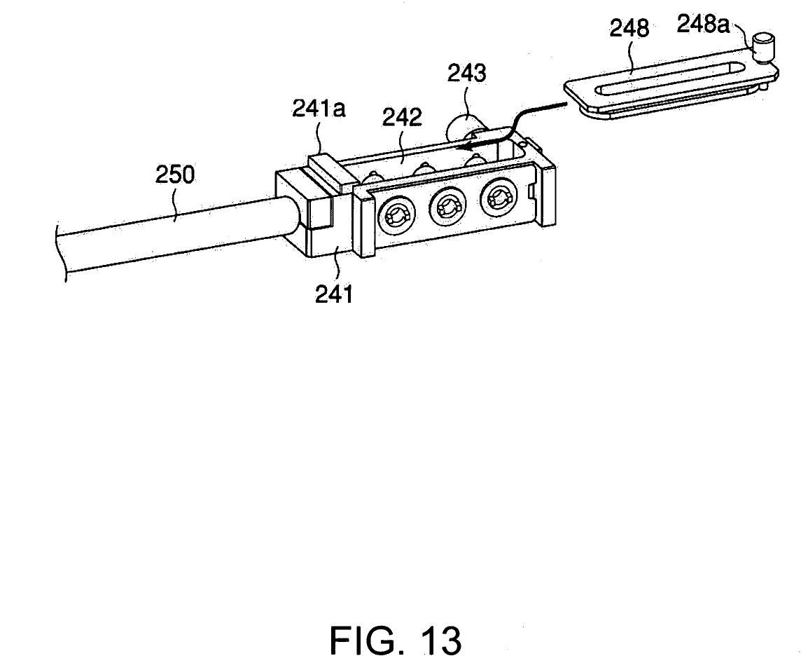

FIG. 13 illustrates an eighth modification of a first non-limiting embodiment of the casing 240. In this example, a wall of the chassis 241 has an opening. The opening is covered with a cover 248. The chassis 241 has a cover mount 241a to catch an end of the cover 248. The cover 248 is clamped on the chassis 241 by a cover screw 248a. The cover 248 includes a groove within which a gasket for keeping an airtight of the casing 240 is fitted. Upon opening the cover 248, washing liquid can run into the cavity 242 from the opening and not only from the flush port 243. Therefore, the washing liquid can flow freely in the cavity 242 and flush the flushing channel 247 well.

In addition, this allows one to actually observe the results of washing in the casing 240.

### NINTH MODIFIED EXAMPLE

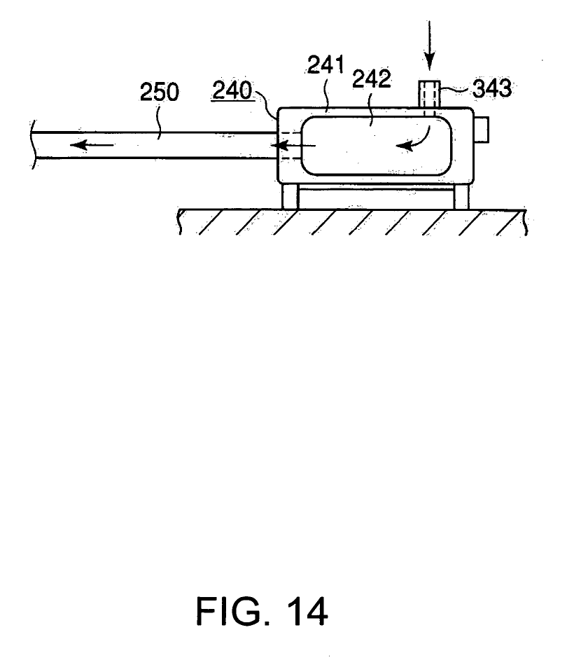

FIG. 14 illustrates a ninth modification of a first non-limiting embodiment of the casing 240. In this example, the flush port 343 is formed on a high position of the chassis 241 and an end of the working unit 220. As a result, washing liquid can run into the cavity 242 from the flush port 343, and can flow all over in the working unit 220, from the flush port 343 to the end effecter 230 through the cavity 242.

### TENTH MODIFIED EXAMPLE

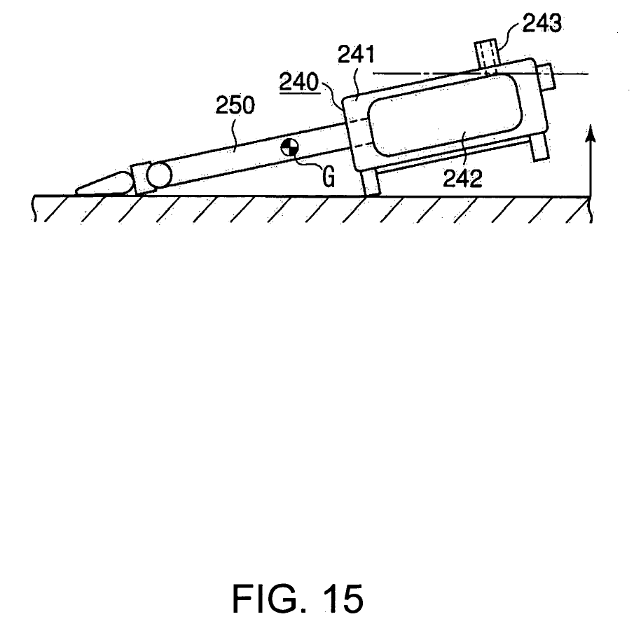

FIG. 15 illustrates a tenth modification of a first non-limiting embodiment of the casing 240. In this example, the end effecter 230 and the connecting unit 250 are both smaller than the casing 240 on a direction perpendicular to an axis of the straight connecting unit 250, and the working unit 220 has a center of gravity G on the connecting unit 250. So, when the working unit 220 is put on the horizontal plane, the end of the connecting unit 250 connected to the end effecter 230 is higher than the other end of the connecting unit 250 connected to the casing 240. The flush port 243 is then in the highest position of the working unit 220.

As a result, the washing liquid flows without resistance of the gravity and the dynamic pressure of the washing liquid from the flush port 243 can be transferred to the other end of the working unit 220. The inside of the working unit 220 can thus be washed well.

### ELEVENTH MODIFIED EXAMPLE

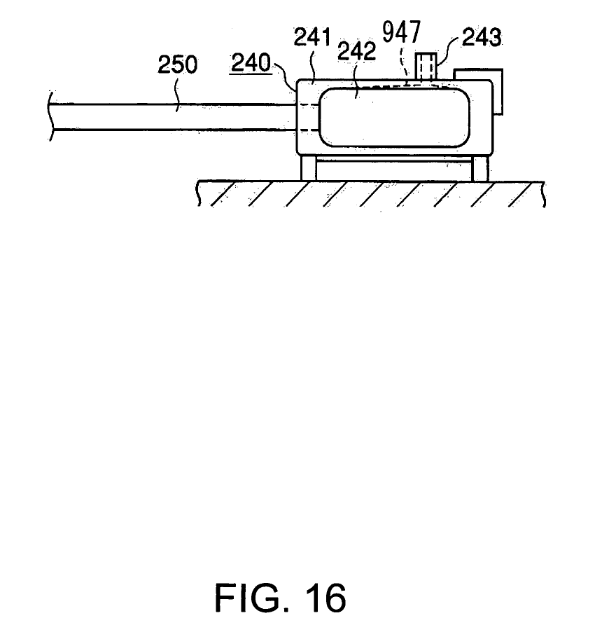

FIG. 16 illustrates an eleventh modification of a first non-limiting embodiment of the casing 240. In this example, the flushing channel 947 is formed deeper near the flush port 243 than far from the flush port 243. As a result, the flow of the washing liquid in the flushing channel 947 becomes smooth.

### TWELFTH MODIFIED EXAMPLE

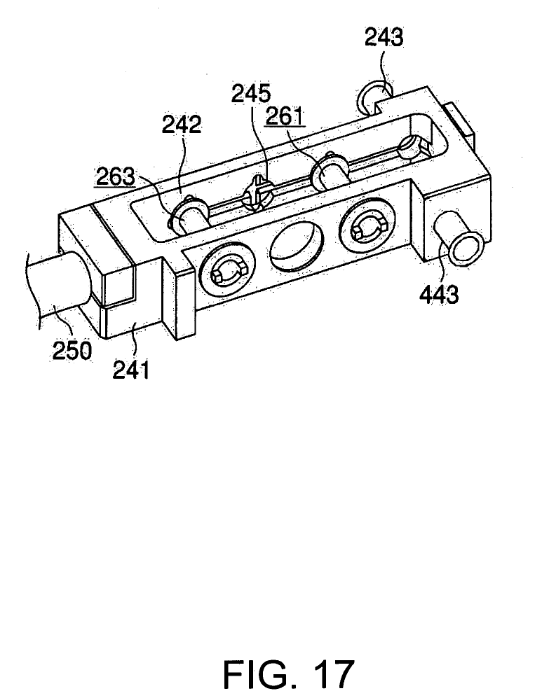

FIG. 17 illustrates a twelfth modification of a first non-limiting embodiment of the casing 240. In this example, the second flush port 443 is formed on the wall opposite the wall on which the first flush port 243 is formed on. As a result, the flow of the washing liquid in the flushing channel 247 becomes smooth.

Embodiments of the present invention could be useful in a variety of medical procedures and are not limited to a laparoscopic surgery. Moreover, the applicability of embodiments of the present invention is not limited to medical procedures involving incisions in the abdominal wall. Possible medical procedures include, but are not limited to, nephrectomy, arthroscopy, gastric bypass or banding, hysterectomy, or any thoracic biopsy.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A medical manipulator comprising:
a rotational assembly configured to transmit a driving force;
a chassis including a cavity configured to house the rotational assembly;
a bearing slot configured to support the rotational assembly;
a flush port connecting an outside of the chassis and the cavity; and
a flushing channel including openings on a shoulder and a bottom of the bearing slot, the openings being open even if the rotational assembly is fitted into the bearing slot.

2. The medical manipulator of claim 1, wherein
the opening of the flushing channel is formed at a position near to the flush port on the shoulder of the bearing slot.

3. The medical manipulator of claim 1, wherein
the opening of the flushing channel is formed on an inside of the flush port.

4. The medical manipulator of claim 1, wherein
the flushing channel is formed parallel to a line through a center of the bearing slot and the flush port.

5. The medical manipulator of claim 1, wherein:
the rotational assembly has a bearing including a collar that contacts the shoulder of the bearing slot; and
the opening of the flushing channel on the shoulder of the bearing slot has a depth along a direction perpendicular to an axis of the bearing slot, the depth being sufficient not to be covered up by the collar.

6. The medical manipulator of claim 1, wherein
the flush port is covered with a lid.

7. The medical manipulator of claim 1, further comprising:
an end effecter configured to be inserted into a patient; and
a connecting unit configured to connect the end effecter and the chassis, including a hollow portion connected to the cavity.

8. The medical manipulator of claim 1, wherein
the flushing channel formed through the shoulder and the bottom of the bearing slot includes at least two openings on the shoulder of the bearing slot.

9. The medical manipulator of claim 1, wherein
the flushing channel is formed perpendicularly to a line through a center of the bearing slot and the flush port.

10. The medical manipulator of claim 1, further comprising:
a second rotational assembly configured to transmit a driving force; and
a second bearing slot configured to support the second rotational assembly, wherein
the flushing channel is formed straightly through the bearing slot and the second bearing slot.

11. The medical manipulator of claim 1, further comprising:
a sub flushing channel configured to cross perpendicularly to the flushing channel.

12. The medical manipulator of claim 1, wherein
the bearing slot is made of a material that has a smaller linear thermal expansion coefficient than that of a material of the chassis.

13. The medical manipulator of claim 1, wherein
a wall of the chassis has an opening covered with a cover.

14. The medical manipulator of claim 1, wherein
the flush port is formed on the highest position of the chassis.

15. The medical manipulator of claim 6, wherein:
the end effecter and the connecting unit are both smaller than the chassis along a direction perpendicular to an axis of the connecting unit; and
a center of gravity of the medical manipulator is on the connecting unit.

16. The medical manipulator of claim 10, wherein
the flushing channel is formed deeper near the flush port than far from the flush port.

17. The medical manipulator of claim 1, further comprising
a second flush port formed on a wall opposite to a wall on which the first flush port is formed.

18. A manipulator for performing a medical procedure, comprising:
an operating unit configured to be operated by an operator, the operating unit comprising an end effecter control unit and a driving unit; and
a working unit comprising an end effecter configured to be inserted in a patient and a flush port configured to allow a thorough cleaning of the working unit.

19. The manipulator of claim 18, wherein the end effecter unit comprises an equipment supported rotatably about at least two axes and a gear set.

20. The manipulator of claim 19, wherein the gear set transmits a driving force to the equipment supported rotatably about at least two axes using a plurality of wires.
